(19) United States
(12) Patent Application Publication
(10) Pub. No.: US 2023/0293827 A1
(43) Pub. Date: Sep. 21, 2023

(54) METHOD AND SYSTEM FOR DETERMINING AN OPTIMAL INSERTION SEGMENT IN A BLOOD VESSEL OF A PATIENT

(71) Applicant: BHEALTHCARE, SAINT-HERBLAIN (FR)

(72) Inventors: Aliaume BRETEAU, Nantes (FR); Jean-Baptiste DE CHAISEMARTIN, Nantes (FR); Sophie BACHELIER, Nantes (FR)

(73) Assignee: BHEALTHCARE, SAINT-HERBLAIN (FR)

(21) Appl. No.: 18/034,690

(22) PCT Filed: Oct. 26, 2021
(86) PCT No.: PCT/EP2021/079632

§ 371 Date: Apr. 29, 2023

(30) Foreign Application Priority Data
Oct. 30, 2020 (FR) FR2011160

(51) Int. Cl. A61M 5/42; G06V 10/44; G06T 7/11

(52) U.S. Cl. CPC A61M 5/427; G06T 7/11; G06V 10/443; G06T 2207/10048; G06T 2207/30101

(57) ABSTRACT

Method for determining at least one optimal insertion segment (810a, 810b, 810c, 810d) in a limb of a patient for inserting a needle into a vein of the patient, said segment (810a, 810b, 810c, 810d) being representative of an insertion point (820a, 820b, 820c, 820d), an insertion direction and a maximum insertion length, comprising a step of near-infrared illumination of the limb of the patient, a step of acquiring near-infrared images of the limb of the patient, a step of pre-processing the acquired images to obtain an image of the veins, a step of applying a linear structure detection filter to said image of the veins to obtain a vascular profile map, a step of binarizing the vascular profile map, a step of skeletonising the veins, a step of defining insertion segments from the skeletons of the veins, a step of classifying the insertion segments according to predetermined classification parameters.

### TECHNICAL FIELD OF THE INVENTION

The invention relates to a method and a system for determining an optimal insertion segment in a blood vessel of a patient, human or non-human. The invention relates more particularly to a method and a system for acquiring and processing images allowing the determination of an optimal insertion segment in a blood vessel of a patient. The invention also relates to an automatic or semi-automatic blood sampling machine using a system in accordance with the invention.

### TECHNOLOGICAL BACKGROUND

Every day, health care facilities perform a very large number of vascular access procedures, for example taking blood, injections, etc. These operations are time-consuming, repetitive and potentially dangerous for the health care personnel and the patient, taking into account the injury risks associated therewith, caused, for example, by the health care personnel having a tremor, being tired, being inexperienced, the patient moving or reacting in an unhelpful manner when the needle is introduced into a blood vessel.

Furthermore, some patients have blood vessels which are not very conducive to the effective taking of blood, which may require several attempts at inserting the needle by the health care personnel before being able to reach a blood vessel permitting the taking of blood. These repeated attempts may be painful for the patient and can cause injuries, which only complicates the sampling operation for these patients.

Moreover, the worldwide coronavirus health crisis requires that systems be developed which limit contacts between patients and the health care personnel and/or which can ensure large-scale screening of the population.

Therefore, the applicant proposed, in WO2015158978 a machine for automatic insertion in a vein of a patient comprising means for capturing a near-infrared image of the arm of the patient, means for detecting a vein in the captured image, a device for holding the detected vein, a needle and means for inserting the needle into the detected vein.

Such a device thus allows blood sampling operations to be automated.

The detection of the vein by this device is a critical step which ensures that the needle is inserted under good conditions in terms of health and safety. In particular, there is a need to find an optimal insertion segment which greatly limits any risk, ensuring that the vein is reached easily. The insertion segment is defined by an insertion point, an insertion direction and a maximum insertion length.

Solutions have been proposed in the prior art for the detection of blood vessels such as veins, in particular for assisting an operator wishing to make a manual insertion by allowing said person to determine which vein appears to be best suited for the insertion of a needle. These solutions use systems for imaging or detecting veins which may be costly, slow and sometimes difficult to use.

However, the prior art solutions are limited to assisting an operator, the operator himself determining the insertion point and the orientation of the needle.

The inventors have thus sought to improve the procedure for determining an optimal insertion segment for allowing in particular automatic and autonomous operation of the choice of the optimal insertion segment in a large number of patients of various profiles, i.e. regardless of their morphology, skin pigmentation, marks, moles, hair, etc.

### AIMS OF THE INVENTION

The invention aims to provide a system and a method for determining an optimal insertion segment in a blood vessel of a patient.

The invention aims in particular to provide a system and a method for automatically and autonomously determining an optimal insertion segment.

The invention aims in particular to provide, in at least one embodiment of the invention, a system and a method for determining which works on a large number of patients of various profiles, i.e. regardless of their morphology, skin pigmentation, marks, moles, hair, etc.

The invention aims in particular to provide, in at least one embodiment of the invention, a system and a method for determining which is effective, inexpensive and easy to implement.

The invention aims in particular to provide, in at least one embodiment of the invention, a system and a method for determining allowing the orientation of the blood vessel to be known and a possible path for the needle during insertion of the needle to be known.

### DESCRIPTION OF THE INVENTION

To this end, the invention relates to a method for determining at least one optimal insertion segment in a blood vessel of a patient for inserting a needle into said blood vessel, said segment being representative of an insertion point in a part of the body of the patient, an insertion direction and a maximum insertion length, comprising the following steps:
a step of illuminating the part of the body of the patient with near-infrared illumination,
a step of acquiring near-infrared images of the part of the body of the patient with at least one camera,
a step of pre-processing the acquired images to obtain an image of blood vessels visible on the surface of the part of the body of the patient, referred to as pre-processed image,
a step of applying a linear structure detection filter to said pre-processed image to obtain an image, referred to as vascular profile map, which identifies the blood vessels visible on the surface of the part of the body of the patient,
a step of binarizing the vascular profile map,
a step of skeletonising the blood vessels on the binarized vascular profile map, configured to obtain, for each blood vessel, a skeleton of said blood vessel,
a step of defining insertion segments from said skeletons of the blood vessels, for each blood vessel,
a step of classifying the insertion segments according to predetermined classification parameters, so as to identify one or more optimal insertion segments.

A method for determining optimum insertion segments in accordance with the invention thus allows the determination of the most favourable insertion segments and allows a classification of these insertion segments to be established in order to guarantee the success and safety in the insertion of the needle in the blood vessel of the patient, by performing processing of the near-infrared image allowing the best characterisation of the subcutaneous vessels. The subcutaneous blood vessels are, for example, veins, arteries, capillaries, depending upon the desired application during insertion of the needle (sampling, injection, etc.).

The different steps of the determining method allow automatic and autonomous determination of the optimal insertion segment, which is effective, inexpensive and easy to implement. Contrary to the solutions of the prior art, the invention is not limited to an aid for a human operator, for example by visualising the location of blood vessels, but the invention allows precise definition of insertion segments, i.e. of an insertion point where the needle should be inserted, an insertion direction, i.e. the axis along which the needle will be inserted, and a maximum insertion length, i.e. the maximum length of the part of the needle which can be inserted, the needle being able to reach the vein within this maximum insertion length based on the orientation of the needle and the depth of the vein. The insertion angle of a needle is generally between 15° and 30° with respect to the surface of the skin, in accordance with current medical practice. The maximum insertion length thus corresponds to the needle length which can be inserted allowing the vein to be reached at the minimum insertion angle. If the needle is inserted at a greater angle, the needle will reach the vein with a shorter insertion length than the maximum insertion length. This precise definition of the insertion segments permits the use of automatic, robotised insertion equipment. By following this segment, the needle will pass through the different layers making up the skin (dermis, epidermis, hypodermis, etc.) until reaching the blood vessel located in the subcutaneous adipose tissue.

The insertion segments can be determined in real time so as to permanently determine the optimal insertion segment(s) as a function of the state of the blood vessels which can vary over time. In particular, the blood vessel may deform or roll under the effect of a mechanical force, for example when inserting the needle.

The pre-processing step allows an image of the blood vessels on the part of the body of the patient to be obtained in order to prepare for the filtering in the following step. In particular, the pre-processing step can comprise the isolation and/or removal, on the image, of irregularities on the skin such as hairs, moles, tattoos, etc., as well as optionally blood capillaries if the desire is to view veins or arteries only. The aim is to enhance the contrast between the subcutaneous blood vessels and the skin of the patient.

The contours of the limb of the patient can also be detected and optionally deleted from the image so as to retain only the data relating to the locations of the blood vessels.

The vascular profile map is obtained via the near-infrared images. In particular, the difference in absorption of the near-infrared rays between the layers of the skin and the haemoglobin (deoxygenated in the veins, oxygenated in the arteries) contained in the subcutaneous blood vessels allows the vascular profile map to be obtained, on which these subcutaneous blood vessels are distinguished from the rest of the patent (skin, muscle, bone, etc.).

The processing performed on the vascular profile map allows use with a large number of patients of various profiles, in particular patients whose veins are difficult to identify directly by sight and/or by touch by palpation of said vein, in particular by a human operator, for example biologist or nurse. The pigmentation of the skin has no bearing on the method in accordance with the invention and so the method is applicable for all phototypes.

The term “near-infrared” is understood to mean a wavelength between 0.7 and 3 µm. This definition corresponds in particular to the infrared ranges IR-A and IR-B as defined by the International Commission on Illumination (CIE). For the detection of blood vessels in a part of the body of a patient, the preferred wavelength interval is between 0.7 and 0.9 µm, in particular for the detection of veins in an arm.

The needle is frequently inserted in a limb of the patient, generally an arm of the patient. The needle is generally inserted in the region of the cubital fossa.

The linear structure detection filter is similar to an edge detection filter or a contour detection filter, allowing the presence of linear structures, in particular blood vessels, to be detected on an image in the invention.

The classification of insertion segments allows the best candidate segment for insertion of the needle to be selected.

According to one variant of the invention, a step of extracting contours of the blood vessels identified in the binarized vascular profile map is performed prior to skeletonising the blood vessels.

Advantageously and in accordance with the invention, the predetermined classification parameters for classifying the insertion segments are selected from one or more parameters from the following list:
the location of the segment with respect to a known pattern of positions of blood vessels on the part of the body of the patient;
the average density of all of the points of the blood vessel included within contours of the blood vessel corresponding to the segment, calculated on the vascular profile map;
the length of the segment;
the depth of the blood vessel in the segment;
the diameter of the blood vessel in the segment;
the orientation of the segment;
the presence or absence of irregularities on the skin on the insertion segment.
a preference of the patient;
a previous insertion history for the same patient.

According to this aspect of the invention, the classification of the insertion segments depends on one or more parameters, optionally weighted, so as to provide optimised determination of the optimal insertion segment, in particular for maximising the chances of success of insertion of the needle and for limiting as well as possible the safety risks.

The parameters and optional weighting of the different parameters can be selected based on several criteria, for example the body mass index (BMI) of the patient, his/her age, the type of needle used, the sampling history of the patient, the sampling history of the insertion machine, the preference of the personnel, the mechanical capabilities of the sampling/injection machine, etc.

The orientation of the segment can be associated in particular with the mechanical capabilities of the machine performing the insertion, because the insertion of the needle at some angles too far away from a nominal working axis of a sampling machine may be impossible to perform (given that the insertion machine is limited for example to an interval between -90° and 90° with respect to a nominal working axis, or a smaller or larger interval and not strictly symmetrical depending on the insertion machines used).

The irregularities of the skin refer in particular to moles, scars, haematomas, petechiae, tattoos, pimples, etc.

Advantageously and in accordance with the invention, the linear structure detection filter is a Frangi filter.

According to this aspect of the invention, the Frangi filter is particularly suitable for detecting blood vessels. It allows precise detection of the blood vessels, which allows, in the following steps, precise skeletonising of the blood vessels, which optimises the determination of insertion segments. The Frangi filter is also independent of the scale used (multi-scale filter), which guarantees effective detection from one patient to another, regardless of the possible profile differences of the blood vessels on the pre-processed image.

According to other variants of the invention, other types of filter or combinations of filters can be used, in particular second derivative filters, etc.

Advantageously and in accordance with the invention, the step of defining insertion segments from skeletons of the blood vessels comprises:
a sub-step of creating a node for each point of each skeleton;
a sub-step of characterising each node to form a graph, a node being a terminal point if it is connected to only a single node, a branch being formed from a set of nodes connected together having only two neighbouring nodes, each branch being weighted by the number of nodes which form it;
a sub-step of verifying each graph by comparing each branch with the corresponding blood vessel on the binarized image;
a sub-step of correcting each non-centred branch on the corresponding blood vessel by dividing the branch into new branches and creating junction nodes between each of the new branches;
a sub-step of defining segments, one segment corresponding to a branch centred on its corresponding blood vessel and having a length greater than a predetermined parameter.

According to this aspect of the invention, the insertion segments are defined by approximation of the skeletons by a graph and by using branches of this graph to form the segments. By using the verifying and correcting sub-steps, which may be performed as many times as necessary, it is ensured that each branch is well centred on the binarized image of the corresponding blood vessel, i.e. it is in the exact centre of the blood vessel, in particular in the centre of the extracted contours of the blood vessel, and having a length sufficiently long to permit insertion of the needle.

These sub-steps allow a first sorting and a listing of the segments which could be used for insertion of the needle, according to their classification following the determining method.

The graphs can comprise one or more nodes connected to at least three branches, which form junction nodes. They reveal the topology of the binary object corresponding to the blood vessel because they allow the retention of a mark of a secondary branch associated with an irregularity or variation in the shape of the object.

Preferably, prior to the verifying sub-step, the step of defining insertion segments from skeletons of the blood vessels comprises a sub-step of simplifying the graph to obtain a final graph, by removing the shortest paths between each terminal point, a longest retained path being the path comprising the most weighted branches. It is this final graph which is used in the following sub-steps. Searching for the shortest segments can be effected using an algorithm for searching for the shortest path, preferably Dijkstra’s algorithm.

Preferably, the sub-step of correcting each non-centred branch comprises:
verifying a criterion for centring the branch on the blood vessel;
searching for critical points of a branch preventing the centring criterion from being met;
dividing the branch point-by-point from the critical point in order to form the new branches, and dividing new branches if necessary;
a new sub-step of correcting each new non-centred branch.

Preferably, the sub-step of defining segments comprises removal of double segments if two segments associated with the same blood vessel are superimposed by more than 65%.

Advantageously and in accordance with the invention, the camera is monochromatic and is equipped with a near-infrared high-pass filter.

According to this aspect of the invention, the images acquired by the camera are directly filtered by the high-pass filter and the vascular profile map is easily obtained via the pre-processing.

The term “camera” is understood to mean any device enabling images to be acquired and enabling the data from these images to be stored and/or transmitted for processing. A camera can be composed of a housing comprising an image acquisition sensor and a lens. The high-pass filter can be arranged on the sensor or on the lens depending upon the embodiment. The camera is configured to pick up at least the wavelengths corresponding to the wavelengths desired for defining the insertion segments, but can be configured to pick up a larger wavelength interval, in which case the high-pass filter enables the desired wavelengths to be targeted.

According to other variants of the invention, the near-infrared filtering is performed digitally on the images obtained by the camera, the camera can be configured to acquire a number of colours (RGB camera), etc.

The method can use images from several cameras, the images thereof being aligned with respect to each other.

The invention also relates to a system for determining at least one optimal insertion segment in a blood vessel of a patient for inserting a needle into said vessel, said segment being representative of an insertion point in a part of the body of the patient, an insertion direction and a maximum insertion length, comprising a unit for acquiring images of the part of the body of the patient and a unit for processing the images acquired by said image acquiring unit,
characterised in that said image acquiring unit comprises:
   near-infrared illumination configured to illuminate the part of the body of the patient with near-infrared illumination, and
   at least one camera configured to acquire near-infrared images of the part of the body of the patient,
and in that the image processing unit comprises:
   a module for pre-processing images configured to be able to provide an image of the blood vessels visible on the surface of the part of the body of the patient, referred to as pre-processed image,
   a module for filtering, configured to apply a linear structure detection filter to said pre-processed image to obtain an image, referred to as vascular profile map, which identifies the blood vessels visible on the surface of the part of the body of the patient,
   a module for binarizing the vascular profile map,
   a module for skeletonising the blood vessels on the binarized vascular profile map, configured to obtain, for each blood vessel, a skeleton of said blood vessel,
   a module for defining insertion segments from said skeletons of the blood vessels, for each blood vessel, and
   a module for classifying the insertion segments according to predetermined classification parameters, configured to identify one or more optimal insertion segments.

Advantageously, the determining system in accordance with the invention implements the determining method in accordance with the invention.

Advantageously, the determining method in accordance with the invention is implemented by the determining system in accordance with the invention.

The invention also relates to an automatic or semi-automatic insertion machine for the insertion of a needle into a part of the body of a patient, for example a limb of the patient, preferably an arm of the patient, comprising a mechatronic assembly such as a robot arm, a unit for controlling said mechatronic assembly, and an insertion head for a needle mounted on the mechatronic assembly, characterised in that it further comprises a determining system in accordance with the invention configured to determine an optimal insertion segment for inserting the needle into the part of the body of the patient.

An automatic or semi-automatic insertion machine equipped with a determining system in accordance with the invention can, in an automatic or semi-automatic and autonomous manner, effect the insertion of the needle on the part of the body of the patient (for blood sampling or injection) based on one of the optimal insertion segments determined by the determining system. The needle can be connected to a syringe, to a catheter, etc.

The insertion machine can be a puncturing or sampling machine if it is intended to collect a blood sample, or an injection machine if it is intended to inject a product into the blood vessel.

The invention also relates to a determining method, a determining system and an insertion machine, which are characterised in combination by all or some of the features mentioned above or below.

### LIST OF FIGURES

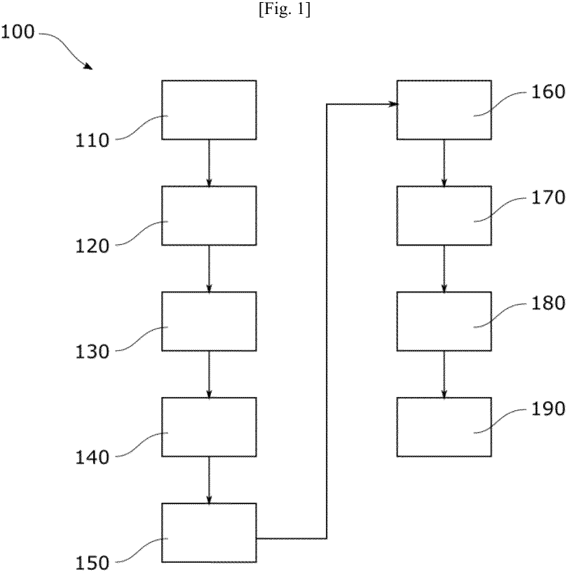

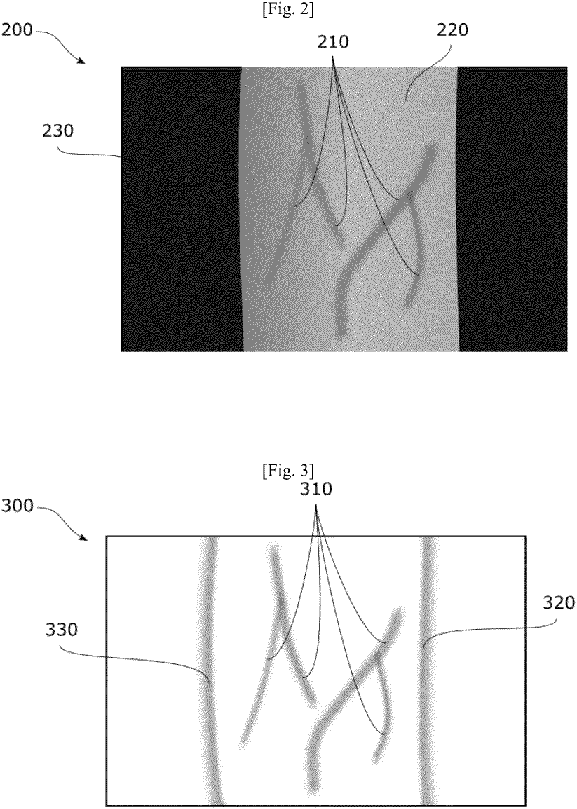

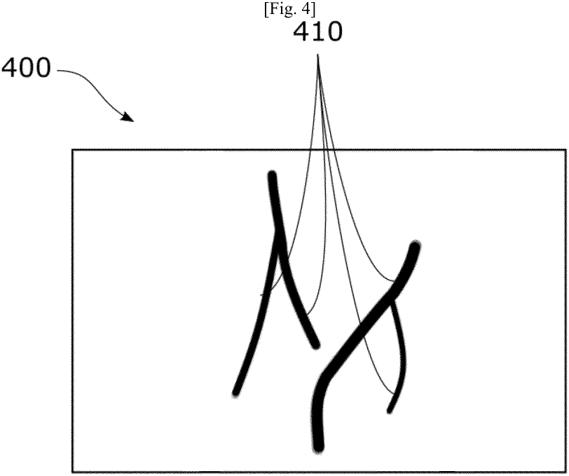

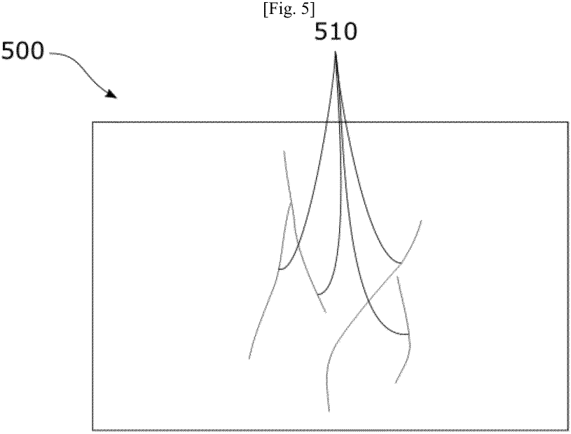

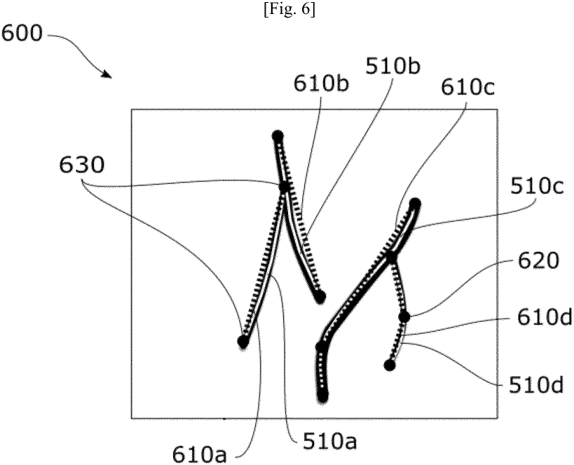

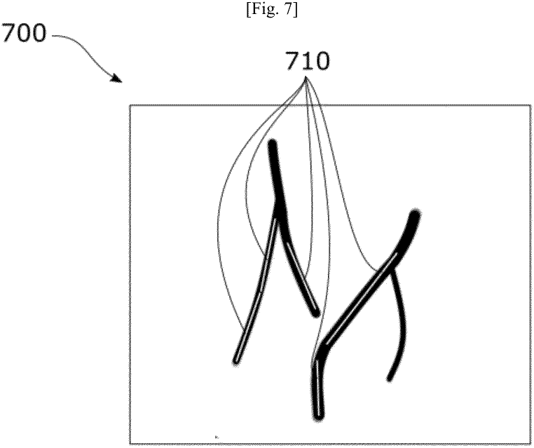

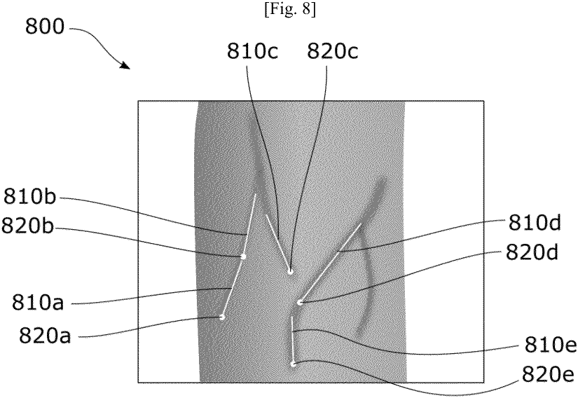

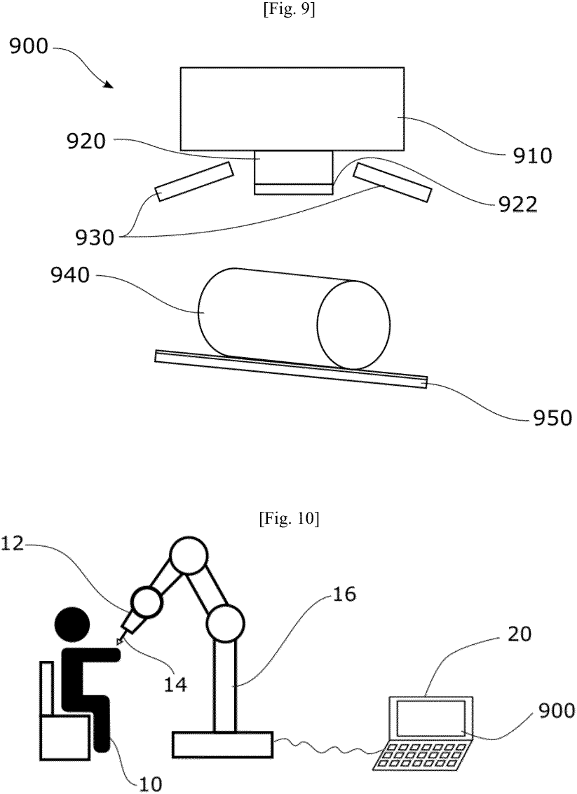

Other aims, features and advantages of the invention will become apparent upon reading the following description given solely in a non-limiting way and which makes reference to the attached figures in which:

[FIG. 1] is a schematic view of a determining method in accordance with one embodiment of the invention,

[FIG. 2] is a representation of a pre-processed photographic image obtained during the implementation of a determining method in accordance with one embodiment of the invention,

[FIG. 3] is a vascular profile map obtained during the implementation of a determining method in accordance with one embodiment of the invention,

[FIG. 4] is a binarized image obtained during the implementation of a determining method in accordance with one embodiment of the invention,

[FIG. 5] is an image representing the skeletonising of the blood vessels, obtained during the implementation of a determining method in accordance with one embodiment of the invention,

[FIG. 6] is an image representing graphs representative of the skeletons of the blood vessels obtained during the implementation of a determining method in accordance with one embodiment of the invention,

[FIG. 7] is an image representing segments from the graphs obtained during the implementation of a determining method in accordance with one embodiment of the invention,

[FIG. 8] is an image representing the determined segments added onto the pre-processed image obtained during the implementation of a determining method in accordance with one embodiment of the invention,

[FIG. 9] is a schematic view of a determining system in accordance with one embodiment of the invention,

[FIG. 10] is a schematic view of an insertion machine in accordance with one embodiment of the invention.

### DETAILED DESCRIPTION OF AN EMBODIMENT OF THE INVENTION

In the figures, for the sake of illustration and clarity, scales and proportions have not been strictly respected.

Furthermore, identical, similar or analogous elements are designated by the same reference signs in all the figures.

FIG. 1 is a schematic view of a determining method in accordance with one embodiment of the invention. The method permits the determination of at least one optimal insertion segment in a part of the body of a patient for inserting a needle into a blood vessel of the patient, said segment being representative of an insertion point in said part of the body of the patient, an insertion direction and a maximum insertion length. The blood vessel is for example a vein, an artery or a capillary.

The method comprises a step 100 of illuminating the part of the body of the patient, for example a limb of the patient, such as in this case an arm of the patient, with near-infrared illumination. The near-infrared illumination is composed of one or more near-infrared lamps. A plurality of near-infrared lamps allow the part of the body of the patient to be illuminated in an homogeneous manner, said part having a volumetric surface which can create shadow zones if the number of lamps is insufficient. The aim is to illuminate the zone of interest uniformly.

The method then comprises a step 120 of acquiring near-infrared images of the part of the body of the patient with at least one camera. The use of the near-infrared illumination associated with the acquisition by the camera allows near-infrared reflectance images to be obtained, referred to as near-infrared spectroscopy.

The method then comprises a step 130 of pre-processing the acquired images to obtain an image of blood vessels visible on the surface of the part of the body of the patient, referred to as pre-processed image. A representation of a pre-processed image obtained by said pre-processing step 130 is, for example, shown with reference to FIG. 2. FIG. 2 corresponds to a representation of a photographic image as captured by a monochromatic camera, then pre-processed. The skin of the limb 220 of the patient has grey shades of variable intensity according to the phototype of the patient and the absorption of the near-infrared rays by the skin. The difference in absorption of the near-infrared rays between the layers of the skin and the haemoglobin contained in the subcutaneous blood vessels allows the blood vessels 210 of the limb 220 of the patient on the pre-processed image 200 to be picked out. A dark background 230 on the pre-processed image 200 enables the edges of the limb 220 to be clearly delimited.

The pre-processing step also comprises processing steps allowing an optimised pre-processed image to be obtained, for example none, one or several of the following processing steps:
thresholding or k-means algorithm to reduce the area to be processed by binarizing the image;
histogram equalisation to accentuate the contrast between the vessels and the skin;
applying a filter to remove the information likely to hamper performance of the following steps, for example a group of hairs on the part of the body of the patient. The applied filter is, for example, a median filter, a Gaussian filter or a bilateral filter.

The method then comprises a step 140 of applying a linear structure detection filter to said pre-processed image to obtain an image, referred to as vascular profile map, which identifies the vessels visible on the surface of the part of the body of the patient. A vascular profile map obtained by said filter-applying step 140 is, for example, shown with reference to FIG. 3. The linear structure detection filter applied in this case is a Frangi filter. The image 300 filtered by said Frangi filter allows just the linear structures, in particular the blood vessels 310 and the contours 320, 330 of the limb of the patient, to be retained in the image.

The method then comprises a step 150 of binarizing the vascular profile map. This step consists of obtaining an image comprising only two pixel values, by thresholding of the luminosities.

The method then comprises, in this embodiment, a step 160 of extracting contours of the blood vessels identified in the binarized vascular profile map; a binarized vascular profile map 400 with blood vessels obtained by said binarization step 150 and contour-extraction step 160 is for example shown with reference to FIG. 4. The contours of the limb of the patient can be deleted from the binarized image so as to retain only the data relating to the locations of the blood vessels 410.

The method then comprises a step 170 of skeletonisation of the blood vessels, for example from the extracted contours, or directly from the binarized vascular profile map, configured to obtain, for each blood vessel, a skeleton of said blood vessel. The skeletons 510 of the blood vessels obtained by said step 170 are for example shown on a skeletonised binary image 500 with reference to FIG. 5. Skeletonising is for example performed by a skeletonisation algorithm, for example morphological skeletonisation or Zhang-Suen skeletonisation. The result of this skeletonising allows skeletons 510 to be obtained, these being a set of curves each describing the centre of the vascular object which has been refined without deterioration of its topology.

The method then comprises a step 180 of defining insertion segments from said skeletons of the blood vessels, for each blood vessel. FIG. 6 schematically shows graphs 610a, 610b, 610c, 610d representative of skeletons of blood vessels, corresponding to the binarized filtered image 600 to show the correspondence of each graph with the contours of the blood vessels and the skeletons. The graphs 610a, 610b, 610c, 610d, in dotted lines, show approximations of the skeletons 510a, 510b, 510c, 510d, shown in white lines. A node is firstly created for each point of the skeleton. Each node is then characterised to form a graph, a node being a terminal point if it is connected to only a single node, a branch being formed from a set of nodes connected together having only two neighbouring nodes, each branch being weighted by the number of nodes which form it. Junction nodes are connected to at least three branches. For example, the graph 610a is an approximation of the skeleton 510a and comprises in particular terminal points 630. The graph 610d is an approximation of the skeleton 510d and comprises in particular a junction node 620.

A branch between two nodes of a graph represents a portion of the substantially rectangular skeleton. However, a branch is an approximation of the skeleton and can sometimes be off-centre with respect to the blood vessel represented by the skeleton, as can be seen for example for graphs 610b and 610d in FIG. 6.

The graph can be simplified to obtain a final graph, by removing the shortest paths between each terminal point, a longest retained path being the path comprising the most weighted branches. It is this final graph which is used in the following sub-steps. Searching for the shortest segments can be effected using an algorithm for searching for the shortest path, preferably Dijkstra’s algorithm.

Processing the graphs thus consists of verifying, in a sub-step of verifying each graph by comparing with the corresponding blood vessel on the binarized image, then correcting each non-centred branch on the corresponding blood vessel by dividing the branch into two new branches and creating a junction node between the two new branches.

In particular, correcting each non-centred branch comprises:
verifying a criterion for centring the branch on the blood vessel;
searching for critical points of a branch preventing the centring criterion from being met;
dividing the branch point-by-point from the critical point in order to form the new branches, and dividing new branches if necessary;
a new sub-step of correcting each new non-centred branch.

Once the branches shorter than a predetermined parameter have been removed and once all the branches are centred, insertion segments are obtained which correspond to said conforming branches. If double segments are obtained, i.e. if two segments associated with the same blood vessel are superimposed by more than 65%, these segments are removed.

These segments 710 can be seen with reference to FIG. 7 representing the segments on a binarized image 700 of the blood vessels. If the insertion segments are restored onto the pre-processed image, an image 800 is obtained representing the determined segments added onto the pre-processed image, as shown in FIG. 8. The insertion segments 810a, 810b, 810c, 810d are characterised respectively by their insertion point 820a, 820b, 820c, 820d, their insertion direction and their maximum insertion length. The insertion point is calculated as the most promising point from among the two ends. The most promising point is for example calculated as a function of the orientation of the part of the body of the patient, the capabilities of the insertion machine, etc. In particular, for insertion into the arm of a patient, the insertion point is generally the one closest to the antecubital fossa.

The method finally comprises a step 190 of classifying the insertion segments according to predetermined classification parameters, so as to identify one or more optimal insertion segments.

FIG. 9 is a schematic view of a determining system in accordance with one embodiment of the invention. The determining system 900 comprises an image processing unit 910 comprising a set of modules configured to implement the determining method described above. One module describes a hardware and/or software brick allowing one or more steps of the method described above to be performed. Several modules can be contained within a single electronic component and/or single piece of software, or the step implemented by one module may require several electronic components and/or pieces of software. The different electronic components can be assembled on a printed circuit board.

The processing unit 910 receives images acquired by an image acquiring unit comprising in particular a near infrared camera 920 and near infrared illumination 930 composed for example in this case of a plurality of light emitting diodes (LEDs) around the camera 920. The camera 920 and the illumination 930 can be controlled by a control module (not shown), for example arranged on the same printed circuit board as one or more modules of the image processing unit 910. The camera 920 can be equipped with a near infrared filter 922. The camera 920 is generally composed of a housing, comprising a sensor, and a lens system comprising individual lenses allowing the focal length and the desired aperture (not shown) to be configured. The near infrared filter 922 can be arranged on the lens or in the housing, according to the embodiments of the invention.

The camera 920 and the illumination 930 are directed towards a part of the body of the patient, in this case a limb 940 of the patient, for example an arm of the patient, represented here by a cylinder of revolution. The arm of the patient is resting on a support 950.

FIG. 10 schematically shows an insertion machine in accordance with one embodiment of the invention. The insertion machine comprises a mechatronic assembly such as a robot arm 16, a unit 20 for controlling the robot arm, a determining system 900 in accordance with the embodiment of FIG. 9, and an insertion head 12. Once the optimal insertion segment has been determined, the control unit 20 transmits, to the actuators of the robot arm 16 and to the actuators of the insertion head 12, the movement information for the needle holder so as to allow insertion of the needle 14 into the part of the body of the patient 10 from which sampling is to be effected.

1. A method for determining at least one optimal insertion segment in a blood vessel of a patient for inserting a needle into said blood vessel, said segment being representative of an insertion point in a part of the body of the patient, an insertion direction and a maximum insertion length, comprising the following steps:
a step of illuminating the part of the body of the patient with near-infrared illumination,
a step of acquiring near-infrared images of the part of the body of the patient with at least one camera,
a step of pre-processing the acquired images to obtain an image of the blood vessels visible on the surface of the part of the body of the patient, referred to as pre-processed image,
a step of applying a linear structure detection filter to said pre-processed image to obtain an image, referred to as vascular profile map, which identifies the blood vessels visible on the surface of the part of the body of the patient,
a step of binarizing the vascular profile map,
a step of skeletonising the blood vessels on the binarized vascular profile map, configured to obtain, for each blood vessel, a skeleton of said blood vessel,
a step of defining insertion segments from said skeletons of the blood vessels, for each blood vessel,
a step of classifying the insertion segments according to predetermined classification parameters, so as to identify one or more optimal insertion segments.

2. The method as claimed in claim 1, wherein the predetermined classification parameters for classifying the insertion segments are selected from one or more parameters from the following list:
the location of the segment with respect to a known pattern of positions of blood vessels on the part of the body of the patient;
the average density of all of the points of the blood vessel included within contours of the blood vessel corresponding to the segment, calculated on the vascular profile map;
the length of the segment;
the depth of the blood vessel in the segment;
the diameter of the blood vessel in the segment;
the orientation of the segment;
the presence or absence of irregularities on the skin on the insertion segment;
a preference of the patient;
a previous insertion history for the same patient.

3. The method of claim 1 as, wherein the linear structure detection filter is a Frangi filter.

4. The method method of claim 1, wherein the step of defining insertion segments from the skeletons of the blood vessels comprises:
a sub-step of creating a node for each point of each skeleton;
a sub-step of characterising each node to form a graph, a node being a terminal point if it is connected to only a single node, a branch being formed from a set of nodes connected together having only two neighbouring nodes, each branch being weighted by the number of nodes which form it;
a sub-step of verifying each graph by comparing each branch with the corresponding blood vessel on the binarized image;
a sub-step of correcting each non-centred branch on the corresponding blood vessel by dividing the branch into new branches and creating junction nodes between each of the new branches;
a sub-step of defining segments, one segment corresponding to a branch centred on its corresponding blood vessel and having a length greater than a predetermined parameter.

5. The method method of claim 1, wherein the camera is monochromatic and equipped with a near-infrared high-pass filter.

6. A system for determining at least one optimal insertion segment in a blood vessel of a patient for inserting a needle into said vessel, said segment being representative of an insertion point in a part of the body of the patient, an insertion direction and a maximum insertion length, comprising a unit for acquiring images of the part of the body of the patient and a unit for processing the images acquired by said image acquiring unit, wherein said image acquiring unit comprises:
near-infrared illumination configured to illuminate the part of the body of the patient with near-infrared illumination, and
at least one camera configured to acquire near-infrared images of the part of the body of the patient,
and in that the image processing unit comprises:
a module for pre-processing images configured to be able to provide an image of the blood vessels visible on the surface of the part of the body of the patient, referred to as pre-processed image,
a module for filtering, configured to apply a linear structure detection filter to said pre-processed image to obtain an image, referred to as vascular profile map, which identifies the blood vessels visible on the surface of the part of the body of the patient,
a module for binarizing the vascular profile map,
a module for skeletonising the blood vessels on the binarized vascular profile map, in order to obtain, for each blood vessel, a skeleton of said blood vessel,
a module for defining insertion segments from said skeletons of the blood vessels, for each blood vessel, and
a module for classifying the insertion segments according to predetermined classification parameters, configured to identify one or more optimal insertion segments.

7. The system as claimed in claim 6, wherein the camera is monochromatic and equipped with a near-infrared high-pass filter.

8. An automatic or semi-automatic insertion machine for the insertion of a needle into a part of the body of a patient, comprising a mechatronic assembly, a unit for controlling said mechatronic assembly, and an insertion head for a needle mounted on the mechatronic assembly, the machine further comprising a determining system, configured to determine an optimal insertion segment for inserting the needle into the part of the body of the patient the determining system comprising:
near-infrared illumination configured to illuminate the part of the body of the patient with near-infrared illumination, and
at least one camera configured to acquire near-infrared images of the part of the body of the patient,
and in that the image processing unit comprises:
a module for pre-processing images configured to be able to provide an image of the blood vessels visible on the surface of the part of the body of the patient, referred to as pre-processed image,
a module for filtering, configured to apply a linear structure detection filter to said pre-processed image to obtain an image, referred to as vascular profile map, which identifies the blood vessels visible on the surface of the part of the body of the patient,
a module for binarizing the vascular profile map,
a module for skeletonising the blood vessels on the binarized vascular profile map, in order to obtain, for each blood vessel, a skeleton of said blood vessel,
a module for defining insertion segments from said skeletons of the blood vessels, for each blood vessel, and
a module for classifying the insertion segments according to predetermined classification parameters, configured to identify one or more optimal insertion segments.
